(19) United States
(12) Patent Application Publication
(10) Pub. No.: US 2016/0067079 A1
(43) Pub. Date: Mar. 10, 2016

(54) NAIL DEFORMITY CORRECTION DEVICE AND MANUFACTURING METHOD FOR NAIL DEFORMITY CORRECTION DEVICE

(71) Applicants: Dong Jun SUN, Suwon-si (KR); Dea II JANG, Seoul (KR); GD Korea CO., LTD., Seongnam-si (KR)

(72) Inventors: Dong Jun SUN, Suwon-si, Gyeonggi-do (KR); Dea Il JANG, Seoul (KR)

(21) Appl. No.: 14/839,509

(22) Filed: Aug. 28, 2015

(30) Foreign Application Priority Data
Sep. 4, 2014 (KR) 10-2014-0118011

(51) Int. Cl. A61F 5/11; A61F 13/02

(52) U.S. Cl. CPC A61F 5/11; A61F 13/02

(57) ABSTRACT

Provided is a nail deformity correction device for effectively correcting a fingernail/toenail by applying an appropriate correction force. The nail deformity correction device includes: a first body having a plate shape, elastically bent by an external force, and being attachable to the surface of a fingernail/toenail; a second body having a plate shape, overlapping the first body, fixed to the first body, and elastically bending with the first body; a core inserted between the first body and the second body and restored into a memorized shape in accordance with a temperature change; and contact portions formed by bringing portions of the first body and the second body in close contact with each other outside the core, in which the first body, the second body, and the core expand and correct a deformed portion of the fingernail/toenail.

### CROSS-REFERENCE TO RELATED APPLICATION

This application claims priority to and the benefit of

Korean Patent Application No. 10-2014-0118011, filed on Sep. 4, 2014, the disclosure of which is incorporated herein by reference in its entirety.

### BACKGROUND OF THE INVENTION

1. Field of the Invention

The present invention relates to a nail deformity correction device for correcting deformed portions of fingernails and toenails, and a method of manufacturing the nail deformity correction device, and more particularly, to a nail deformity correction device that effectively correct fingernails and toenails without a side effect such as pain of a user by providing an appropriate correction force, and a method of manufacturing the nail deformity correction device which an more conveniently and easily manufacture the nail deformity correction device.

2. Description of the Related Art

Fingernails or toenails, which are portions of the cuticles transformed into horny tissues, cover and protect weak inside skins of fingers or toes. Fingernails or toenails are made from Keratin that is one of hard fibrous proteins and can prevent the underlying tissues in close contact with fingers or toes. In a normal state, fingernails or toenails grow outward from the ends of fingers or toes.

However, when fingers or toes have various diseases, fingers or toes are partially deformed by internal or external causes, or a person has a bad habit having a bad influence on fingernails or toenails, fingernails or toenails may abnormally grow and deform. In this case, the fingernails and toenails have difficulty in maintaining their normal protection function and cause pains by pressing the weak inside skins. In particular, ingrown nails that abnormally bending into cuticles cause severe pains and make normal life difficult.

Accordingly, correction devices for correcting deformed fingernails or toenails into the normal state have been used in the related art. A correction device may be configured, for example to press affected parts of a deformed fingernail or toenail on the nails, thereby straightening the deformed fingernail or toenail in the normal state. For example, a clip-shaped bending device for correcting fingernails or toenails has been disclosed in Korean Patent No. 10-0720646.

However, the device is difficult to mount, so individuals have difficulty in using the device. Further, the device presses an affected part of a user too much, so it causes pains or cannot provide an appropriate level of correction force, and accordingly, its correction effect is not sufficient. That is, the device cannot provide an appropriate level of correction force suitable for the state of a user, so it is difficult to correct an affect part well. Further, correction devices of the related art provides the same level of correction force even though affected parts have various sizes, positions, and shapes, so they are not useful.

### CITATION LIST

### Patent Literature

Patent Literature 1: Korean Patent No. 10-0720646 (May 21, 2007), FIG. 1, FIG. 5, and FIG. 10

### SUMMARY OF THE INVENTION

The present invention has been made in an effort to solve the problems and an object of the present invention is to provide a nail deformity correction device that can effectively correct fingernails and toenails without a side effect such as a pain of a user by providing an appropriate correction force, and a method of conveniently and easily manufacturing the nail deformity correction device.

The objects of the present invention are not limited to those described above and other objects may be made apparent to those skilled in the art from the following description.

A nail deformity correction device of the present invention includes: a first body having a plate shape, elastically bent by an external force, and being attachable to the surface of a fingernail/toenail; a second body having a plate shape, overlapping the first body, fixed to the first body, and elastically bending with the first body; a core inserted between the first body and the second body and restored into a memorized shape in accordance with a temperature change; and contact portions formed by bringing portions of the first body and the second body in close contact with each other outside the core, in which the first body, the second body, and the core expand and correct a deformed portion of the fingernail/toenail, and at least a portion of the contact portion resists restoration of the core.

The contact portions may have curvature-maintaining portions expanding outward from ends of the core.

The core may memorize a shape to expand straight in a longitudinal direction at a set temperature and the curvature-maintaining portions are adjusted to maintain a predetermined curvature against transformation of the core.

The curvature of the core may increase in proportion to the area of the curvature-maintaining portions.

The contact portions may have curved portions convexly protruding outward at ends, and the ratio ‘b/a’ between a straight line ‘a’ connecting the start point and the end point of the curved portions and a perpendicular line ‘b’ from the top of the curved portions to the straight line ‘a’ may be less than ½.

The contact portions may have a shape in which convex portions and concave portions are continuously connected.

The first body may be bonded to the fingernail/toenail by an adhesive and an attachment surface being in close contact with the adhesive may be formed on the side of the first body facing the fingernail/toenail.

The attachment surface may have prominences and depressions thereon.

The adhesive may be hardened and fixed to the attachment surface and may include thermoplastic resin that has fluidity when being heated.

The core may be made of a shape memory alloy that memorizes the shape to expand straight at 40° C.

The first body and the second body may include polymeric resin having elasticity and plasticity.

The first body and the second body may each have a receiving portion formed on the surface facing the core and the core may be inserted between the receiving portions; at least one of fusion grooves and fusion protrusions may be formed outside the receiving portions and the fusion grooves and the fusion protrusions formed outside the different receiving portions may be combined with each other; and the first body and the second body may be melted and fixed.

A portion of the core may have an exposing portion that is exposed to the outside.

The contact portions may extend from an end of the core and the exposing portion may be formed at the other end of the core.

A method of manufacturing a nail deformity correction device according to the present invention includes: (A) preparing a mold having first grooves formed in a plate shape, second grooves formed in a plate shape corresponding to the first grooves, and at least one resin injection passage connected to the first grooves and the second grooves; (B) forming a first body having a plate shape corresponding to the first grooves and a second body having a plate shape corresponding to the second grooves by injecting and hardening resin in the first grooves and the second grooves through the resin injection passage; (C) separating the first body and the second body from the mold and inserting and aligning a core made of a shape memory material between the first body and the second body; and (D) attaching the first body and the second body with the core between the first body and the second body.

The resin injection passages in the step (A) and the step (B) may be positioned in the same plane as at least one of the first grooves formed in a plate shape and the second grooves formed in a plate shape and connected in parallel with at least one of the first grooves and the second grooves.

The first body and the second body in the step (C) may be fixed by runner connecting portion formed by the resin hardened in the resin injection passages in the step (B), and the runner connecting portions may be positioned in the same plane as at least one of the first body having a plate shape and the second body having a plate shape and connected in parallel with at least one of the first body and the second body.

In the step (A), the first grooves and the second grooves may be formed in separate pairs on mold surfaces facing each other of different mold frames constituting the mold by being separably combined.

In the step (C), the first body and the second body may each have a receiving portion formed on the sides facing the core and the core may be inserted between the different receiving portions.

In the step (C), the first body and the second body may further have at least one of fusion grooves formed outside the receiving portions and fusion protrusions formed outside the receiving portions, and the fusion grooves and the fusion protrusions formed outside the different receiving portions may be combined and aligned.

The attachment in the step (D) may be achieved by melting the fusion grooves and the fusion protrusions fitted in the fusion grooves and bonding the first body and the second body to each other.

In the step (C), at least one of the first body and the second body may further have linear fusion portions extending along the edge of the receiving portion and the fusion portions may be positioned between the first body and the second body.

The attachment in the step (D) may be achieved by melting the fusion portions and bonding the first body and the second body to each other.

A contact surface having prominences and depressions may be formed in the first grooves in the step (A) and prominences and depressions may be formed on a surface of the first body which corresponds to the contact surface.

The method may further includes a step of forming a cutting surface such that cross-sections of the first body, the second body, and the core are all exposed, and separating the first body, the second body, and the core along the cutting surface, after the step (D).

### BRIEF DESCRIPTION OF THE DRAWINGS

FIG. 1 is a perspective view showing a nail deformity correction device according to an embodiment of the present invention;

FIG. 2 is a cross-sectional view showing the nail deformity correction device shown in FIG. 1, taken in the longitudinal direction;

FIG. 3 is a plan view and a bottom view of the nail deformity correction device of FIG. 1;

FIG. 4 is a cross-sectional view showing a modified example of the nail deformity correction device of FIG. 1;

FIG. 5 is a plan view showing another modified example of the nail deformity correction device of FIG. 1;

FIGS. 6 to 8 are views illustrating a restoration process and a correction force adjustment process of the nail deformity correction device;

FIGS. 9 and 10 are views showing a process of mounting the nail deformity correction device of FIG. 1;

FIGS. 11 to 13 are view showing a process of correcting a deformed fingernail or toenail with the nail deformity correction device of FIG. 1;

FIG. 14 is a cross-sectional view showing a nail deformity correction device according to another embodiment of the present invention, taken in the longitudinal direction;

FIGS. 15 and 16 are views showing a process of mounting the nail deformity correction device of FIG. 14;

FIG. 17 is a perspective view showing an example of an available mold prepared in accordance with a method of manufacturing a nail deformity correction device according to an embodiment of the present invention;

FIG. 18 is a perspective view showing a process of injection resin with the mold of FIG. 17 assembled;

FIG. 19 is a perspective view showing primary separation with a first body and a second body of the mold of FIG. 18 connected;

FIG. 20 is an enlarged perspective view showing the first body and the second body of FIG. 19 that are completely separated;

FIGS. 21 and 22 are views showing a process of injecting and arranging a core in between the first body and the second body of FIG. 20;

FIG. 23 is a view showing a process of attaching the first body and the second body of FIG. 22;

FIG. 24 is a perspective view showing an example of a nail deformity correction device formed by attaching the first body and the second body of FIG. 23 with the core therebetween; and

FIG. 25 is a view showing a process of disassembling the nail deformity correction device of FIG. 24.

### DETAILED DESCRIPTION OF THE PREFERRED EMBODIMENTS

The advantages and features of the present invention, and methods of achieving them will be clear by referring to the exemplary embodiments that will be describe hereafter in detail with reference to the accompanying drawings. However, the present invention is not limited to the exemplary embodiments described hereafter and may be implemented in various ways, and the exemplary embodiments are provided to complete the description of the present invention and let those skilled in the art completely know the scope of the present invention and the present invention is defined by claims. Like reference numerals indicate the same components throughout the specification.

In the following description, a fingernail or a toenail is generally referred to as ‘fingernail/toe nail’ or ‘nail’. For example, a ‘deformed affected part of a fingernail/toenail’ means an affected part due to deformation of any one of a fingernail and a toenail and ‘mounting a nail deformity correction device on a fingernail/toenail’ means mounting a nail deformity correction device on any one of a fingernail and a toenail to correct an affected part.

A ‘longitudinal direction of a fingernail/toenail’ means a general growth direction of a fingernail or a toenail from a finger of a toe. On the other hand, a ‘width direction’ of a fingernail/toenail is a direction perpendicular to the general growth direction of a fingernail or a toenail and a deformed affected part of a fingernail/toenail may be usually caused by width-directional bending of a fingernail/toenail.

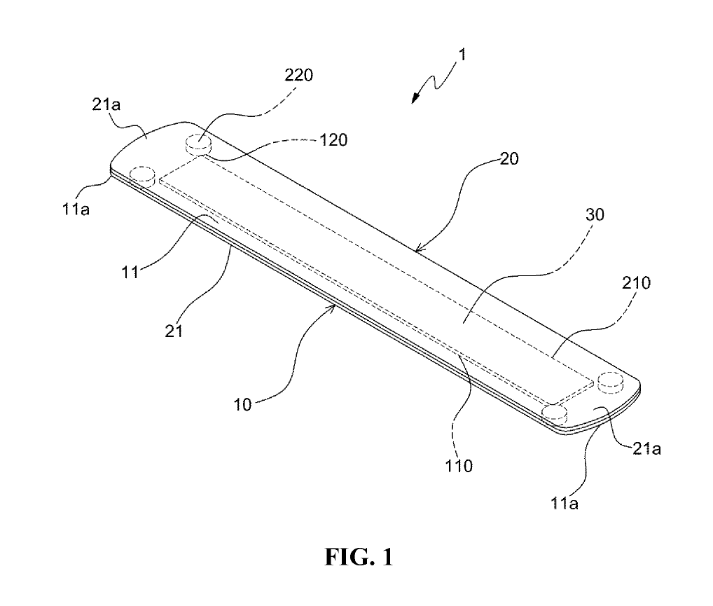

Hereinafter, a protective case for a nail deformity correction device according to an embodiment of the present invention is described with reference to FIGS. 1 to 13.

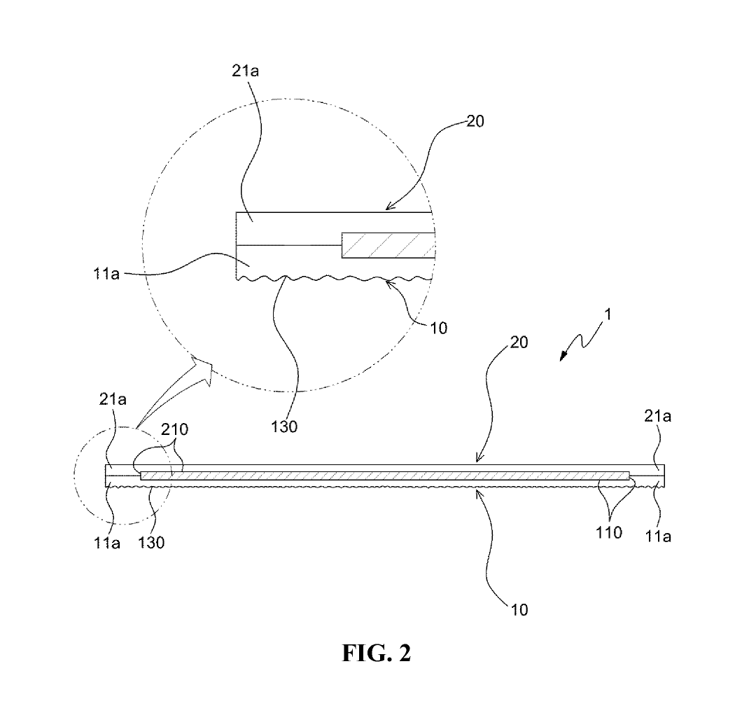

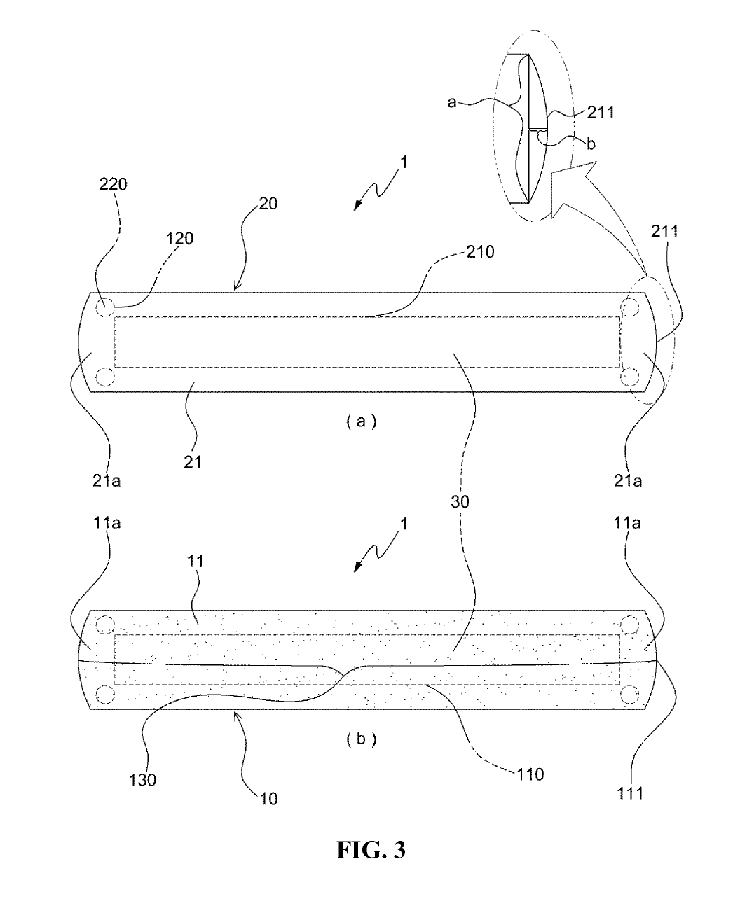

FIG. 1 is a perspective view showing a nail deformity correction device according to an embodiment of the present invention, FIG. 2 is a cross-sectional view showing the nail deformity correction device shown in FIG. 1, taken in the longitudinal direction, and FIG. 3 is a plan view and a bottom view of the nail deformity correction device of FIG. 1. (a) of FIG. 3 is a plan view and (b) of FIG. 3 is a bottom view.

Referring to FIGS. 1 to 3, a nail deformity correction device 1 according to an embodiment of the present invention includes a first body 10 to be attached to the surface of a fingernail/toenail, a second body 20 overlapping the first body 10 and fixed to the first body 10, a shape memory core 30 inserted in between the first body 10 and the second body 20 and memorizing a shape to be restored into the memorized shape in accordance with a temperature change, and contact portions 11 and 21 formed by partially bringing the first body 10 and the second body 20 in close contact with each other on the core 30. The core 30 memorizes a shape to be restored into the memorized shape in accordance with a temperature change, the first body 10 is elastically bent by an external force, and the second body 20 is also elastically bent with the first body 10. Accordingly, as the core 30 is restored, the first body 10, the second body 20, and the core 30 are deployed on a deformed portion of a fingernail/toenail to correct it, and the contact portions 11 and 21 expands outward from the core 30 and at least partially resists restoration of the core 30.

That is, in the nail deformity correction device 1 according to an embodiment of the present invention, the first body 10 and the second body 20 are expanded with the core 30 to correct a fingernail/toenail by restoration or restoration force of the core 30, and particularly, the first body 10 and the second body 20 being in close contact with each other outside the core 30 at least partially resist the restoration force, so the degree of expansion of the core 30 or the entire nail deformity correction device 1 including the core 30 can be adjusted. Accordingly, it is possible to maintain a correction force (it means the magnitude of a resultant force applied to a deformed portion of a fingernail/toenail and may be defined as combination of elasticity and plasticity of the first body and the second body, contact force between the contact portions, hardness increased by the contact, resistant due to attachment force of the contact portions for maintaining the attachment to a fingernail/toenail) applied to a deformed affected portion at an appropriate level.

Accordingly, the nail deformity correction device 1 can prevent a side effect such as causing a pain by excessively pressing a fingernail/toenail and effectively correct an affected part by apply an optimal correction force. Further, the nail deformity correction device can increase the effect of correcting a fingernail/toenail without separating by being widely and efficiently attached to a fingernail/toenail and the core 30 can be restored at a set temperature such that easier correction is possible in accordance with the user's environmental circumstance. The nail deformity correction device 1 having these characteristics is described in more detail with reference to the drawings.

The first body 10 is formed in the shape of a plate. The first body 10 may be formed in the shape of a plate elongated in one direction, as shown in FIGS. 1 to 3. The first body 10 may be fixed to the second body 20, overlapping it, and can be elastically bent by an external force by being made of polymeric resin having elasticity and plasticity. The polymeric resin may be polyamide resin. The relative magnitudes of the elasticity and plasticity and the magnitude of strength of the first body 10 may be appropriately changed by adjusting the component ratio of the material, so it can be easily expanded with the core 30 and resist restoration of the core 30 at the portion (for example, the contact portion) where contact with the core 30 is minimized.

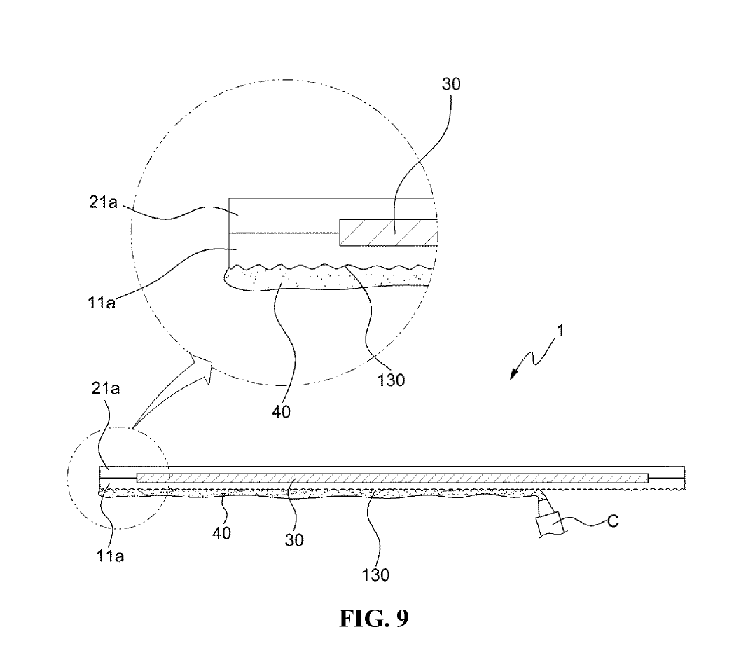

The first body 10 may be bonded to a fingernail/toenail by an adhesive (see 40 in FIG. 9) and the side of the first body 10 facing a fingernail/toenail may be an attachment surface 130 that is brought in close contact with the adhesive 40, as shown in FIG. 2. The attachment surface 130 may have prominences and depressions and the adhesive 40 permeates in between the fine prominences and depressions on the attachment surface 130, so a more firm bonding layer can be formed between a fingernail/toenail and the nail deformity correction device 1. The attachment surface 130, as shown in (b) of FIG. 3, may be formed throughout the side facing a fingernail/toenail of the first body 10, that is, throughout the bottom of the nail deformity correction device 1. However, the attachment surface 130 is not limited to the configuration with prominences and depressions and may be surface-finished such as forming protrusions or a pattern such that the adhesive 40 can be easily bonded.

The second body 20 is also formed in the shape of a plate. The second body 20 may be formed in a shape corresponding to the first body 10 to overlap the first body 10 in a pair. The second body 20 overlaps the first body 10 and is fixed to the first body 10, and is elastically bent with the first body 10. The second body 20 may also be made of polymeric resin having elasticity and plasticity and the polymeric resin may be polyamide resin. The relative magnitudes of the elasticity and plasticity and the magnitude of strength of the second body 20 may also be appropriately changed by adjusting the component ratio of the material, so it can be easily expanded with the core 30 and resist restoration of the core 30 at the portion (for example, the contact portion) where contact with the core 30 is minimized.

The first body 10 and the second body 20 are formed in an oblong shape, but in detail, they may have at least partially a curved shape. For example, curved portions (see 111 and 211 in FIG. 3) may be formed at the contact portions 11 and 21 and the contact portions 11 and 21 or the entire nail deformity correction device 1 can be more stably attached to a fingernail/toenail by the curved portions 111 and 211 having appropriate shapes. Further, the first body 10 and the second body 20 may be firmly fixed to each other by fusing with the core 30 therebetween and may be more easily aligned and attached by coupling structures such as fusion grooves 120 and fusion protrusions 220. This will be described in more detail below.

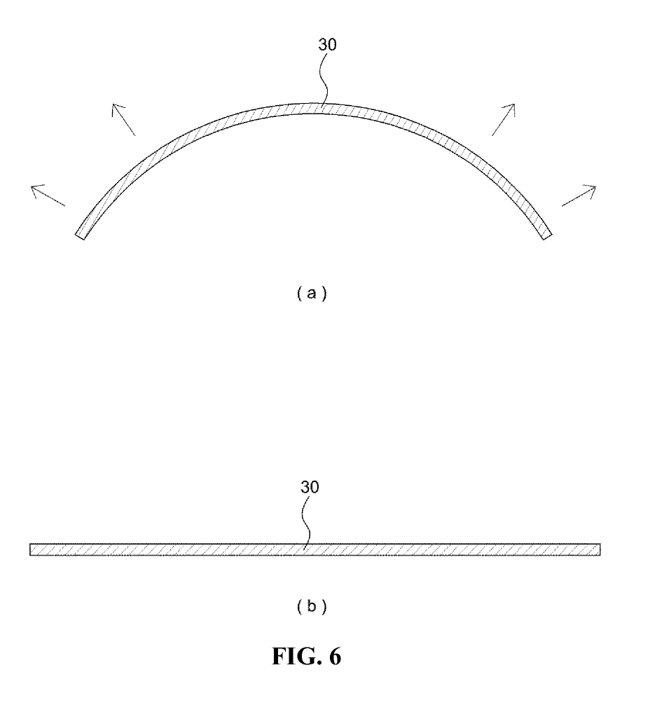

The core 30 is inserted between the first body 10 and the second body 20, as shown in FIGS. 1 to 3. The core 30 memorizes a shape to be restored into the memorized shape in accordance with a temperature change and it can memorize a shape to extend straight longitudinally at a set temperature (see (b) of FIG. 6). Accordingly, the entire nail deformity correction device 1 attached to a deformed fingernail/toenail can be expanded with the core 30 and correct an affected part into a normal state. The core 30, as shown in the figures, may be formed in the shape of a plate narrower than the first body 10 and the second body 20, but it is not limited thereto and may be modified in various shapes. For example, the core 30 may be elongated in one direction such that a cross-section perpendicular to the elongation direction has a circular shape, an elliptical shape, or other various shapes. The core 30, for example, may be a shape memory alloy.

The first body 10 and the second body 20 have receiving portions 110 and 210 on the sides facing the core 30, as shown in FIG. 2, so the core 30 can be inserted between the receiving portions 110 and 210. That is, as shown in FIGS. 1 to 3, the core 30 can be inserted and firmly fixed in between the receiving portion 110 recessed on the first body 10 and the receiving portion 210 recessed on the second body 20. Further, any one of the fusion grooves 120 and the fusion protrusions 220 may be formed outside the receiving portions 110 and 210, as shown in FIGS. 1 and 3, so the fusion grooves 120 and the fusion protrusions 220 formed outside the different receiving portions 110 and 220 can be coupled to each other. That is, the fusion grooves 120 are formed on any one of the first body 10 and the second body 20 outside the receiving portions 110 and 210 and the fusion protrusions 220 are formed on any one of the first body 10 and the second body 20, at positions corresponding to the fusion grooves 120, so the first body and the second body can be firmly fixed by coupling the fusion grooves 120 and the fusion protrusions 220.

As described above, the nail deformity correction device 1 can be more firmly manufactured by forming the structures (receiving portions, fusion grooves, fusion protrusions etc.) that make it easy to receive the core 30 between the first body 10 and the second body 20 or combine the first body 10 and the second body 20. The second body 20 and the first body 10 may be fixed by fusing, as described above, and particularly, the portions or sides where the first body 10 and the second body 20 are melted by heat such as the fusion grooves 120 and the fusion protrusions 220, so the first body and the second body can be melted and firmly fixed. Various structures that can be melted and bonded by heat other than the fusion grooves 120 and the fusion protrusions 220 may be formed at the portion where the first body 10 and the second body 20 are in contact with each other.

The contact portions 11 and 21 are formed by partially bringing the first body 10 and the second body 20 in contact with each other outside the core 30. The contact portions 11 and 21, as shown in FIGS. 1 and 3, are the parts extending outside the core 30 and being in direct or minimally contact with the core 30, and as described above, they may be portions of the first body 10 and the second body 20 being melted and firmly in close contact with each other. Accordingly, even though the core 30 is restored by a strong restoration force at a set temperature, the contact portions 11 and 21 can resist the restoration of the core 30, maintaining its shape.

That is, the contact portions 11 and 21 provide the resistant force against the restoration, so the entire nail deformity correction device 1 can be expanded straight. Accordingly, the correction force applied to a fingernail/toenail by the nail deformity correction device 1 is maintained at an appropriate level. For example, when the nail deformity correction device 1 is attached to a fingernail/toenail, all of the first body 10, the second body 20, and the core 30 bend into a shape corresponding to the shape of the fingernail/toenail. The contact portions 11 and 21 being in close contact with each other with smallest contact with the core 30 outside the core 30 keep bending by a combination of the elasticity and plasticity of the contact portions 11 and 21, which are portions of the first body 10 and the second body 20, the contact force between the contact portions 11 and 21, hardness increased with transformation of the sides when they are in contact with each other by fusing, and attachment force for keeping the contact portions 11 and 21 attached to the fingernail/toenail. Accordingly, even though the core 30 is restored and expanded by a strong restoration force at a set temperature, the contact portions 11 and 21 minimize a change of their shapes against the restoration of the core 30, the entire nail deformity correction device 1 can maintain a predetermined curvature.

In particular, the contact portions 11 and 12 have curvature-maintaining portions 11a and 21a expanding outward from the ends of the core 30, as shown in FIGS. 1 to 3. The curvature-maintaining portions 11a and 21a are positioned outside further than the core 30 in the expanding direction of the core 30, so they can more easily resist the restoration force of the core 30. Accordingly, the core 30 memorizes a shape to expand straight in the longitudinal direction at a set temperature and the curvature-maintaining portions 11a and 21a can maintain a predetermined curvature against the shape change of the core 30. Further, the resistance increases and the curvature of the core 30 increases in proportion to the areas of the curvature-maintaining portions 11a and 21a, so it is possible to change the curvature of the core 30 and the magnitude of the correction force applied to a fingernail/toenail by adjusting the size or the ratio of the curvature-maintaining portions 11a and 21a. This will be described in more detail below.

The contact portions 11 and 21 may have curved portions 111 and 211 protruding convexly outward at the ends, described above. The contact portions 11 and 21 have the curved portions 111 and 211, and as shown in FIG. 3, and the ratio ‘b/a’ between a straight line ‘a’ connecting the start point and the end point of the curved portions 111 and 211 and a perpendicular line ‘b’ from the top of the curved portions 111 and 211 to the straight line ‘a’ may be less than ½. As described above, in order that the curved portions 111 and 211 make a continuous curved line and maintain an appropriate curvature, the area of the entire contact portions 11 and 21 attached to a fingernail/toenail can be increased by the convex portion and the contact portions 11 and 21 can be attached maximally uniformly onto a fingernail/toenail making a curved surface.

When the ratio ‘b/a’ is too large, the curvature of the curved portions 111 and 211 are excessively increased or the curved portions 111 and 211 are transformed to be pointed, so the size of the attachment surface may decrease and the attachment force may be inappropriately applied to a fingernail/toenail. Accordingly, by forming the smooth curved portions 111 and 211 with the ratio, the attachment force can be increased. Further, by appropriately adjusting the ratio ‘b/a’ in consideration of the curved degree of the surface of a deformed fingernail/toenail or the curved surface of a fingernail/toenail, the difference between the convex portion and the other portion of the curved portions 111 and 211 decreases when it is in close contact with the fingernail/toenail, so the attachment force can be more uniformly applied to the fingernail/toenail.

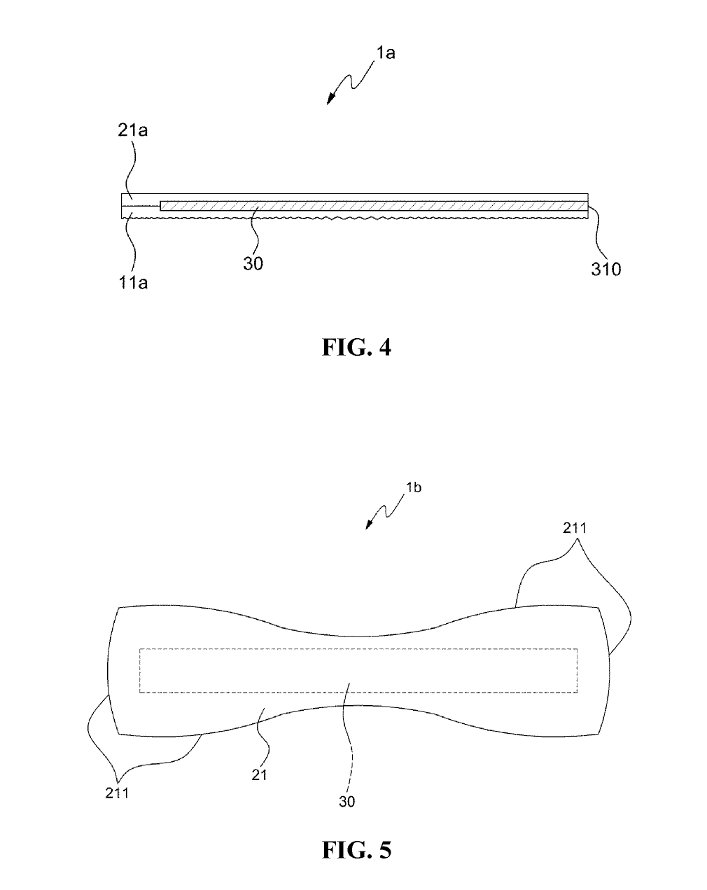

FIG. 4 is a cross-sectional view showing a modified example of the nail deformity correction device of FIG. 1 and FIG. 5 is a plan view showing another modified example of the nail deformity correction device of FIG. 1.

As shown in FIG. 4, a nail deformity correction device 1a may be modified to have an exposing portion 310 that partially exposes the core 30 to the outside. The contact portions, particularly, the curvature-maintaining portions 11a and 21a extend from an end of the core 30 and the exposing portion 310 may be formed at the other end of the core 30. In this case, the curvature-maintaining portions 11a and 21a and the contact portions are not formed outside the portion where the exposing portion 310 is formed, so the nail deformity correction device 1a is restored in an asymmetric shape and different correction forces can be applied to a fingernail/toenail at different positions. This will be described in more detail below.

Further, as shown in FIG. 5, the nail deformity correction device 1b may be modified into a shape in which a contact portion 21 having a convex portion and a concave portion continuously connected to each other. In this case, curve portions 211 may be repeatedly formed along the edge of the contact portion 21 in a connected or separated shape and the separated portions between the curved portions 211 are recessed such that the convex portions and the concave portion are continuously connected. Accordingly, it is possible to adjust the attachment position or the attachment area of the contact portion 21 formed outside the core 30, corresponding to a deformed shape of a fingernail/toenail and to attach the nail deformity correction device 1b optimally to them.

In addition, as the nail deformity correction device 1b is variously change in shape, it is possible to make a user feel like not being treated as a patient, but being corrected simply with not a beauty tool by providing the nail deformity correction device 1b in an esthetic shape familiar to the user such as a ribbon.

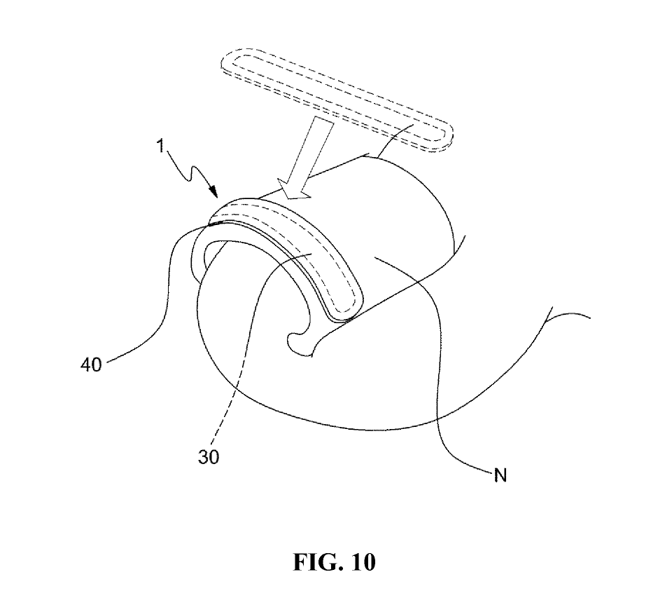

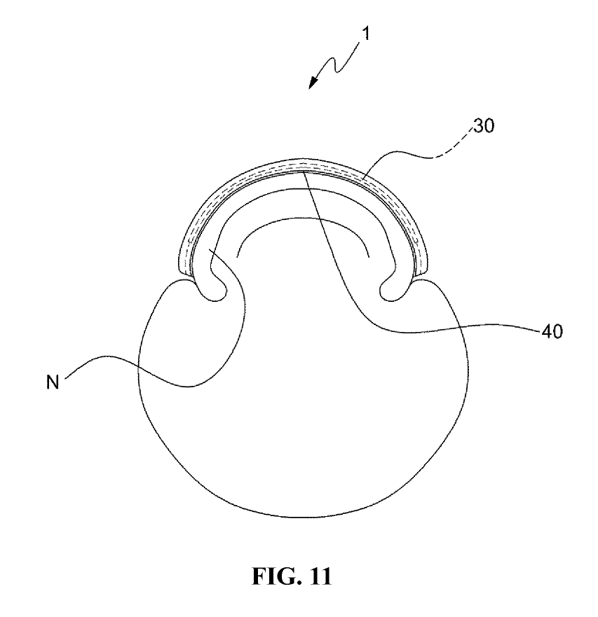

FIGS. 6 to 8 are views illustrating a restoration process and a correction force adjustment process of the nail deformity correction device, FIGS. 9 and 10 are views showing a process of mounting the nail deformity correction device of FIG. 1, and FIGS. 11 to 13 are view showing a process of correcting a deformed fingernail or toenail with the nail deformity correction device of FIG. 1.

Hereafter, a restoration process and a correction force adjustment process of the nail deformity correction device according to an embodiment of the present invention and a process of correcting a deformed fingernail or toenail with the nail deformity correction device are sequentially described with reference to FIGS. 6 to 13.

First, a process of supplying and adjusting a correction force due to restoration of the nail deformity correction device is described with reference to FIGS. 6 to 8. Referring to FIG. 6, as described above, the core 30 bends in a shape corresponding to the shape of a fingernail/toenail, as in (a) of FIG. 6, and then when the temperature changes, it is expanded and restored into the memorized shape, as in (b) of FIG. 6. FIG. 6 shows only the core 30 for helping understanding. The core 30, as shown in (b) of FIG. 6, may memorize the shape to be expanded straight in the longitudinal direction at a set temperature. For example, the core 30 may be a shape memory alloy that expands straight at 40° C.

Since a fingernail/toenail can more flexibly deform when it is dry than wet, if the core 30 memorizes the shape to expand when a user take shower, more efficient correction effect can be achieved. For example, considering that the temperature of a shower booth is about 36° C., more correction effect can be expected by making the core 30 expand at 40° C. However, it is just an example, and the temperature where the core 30 is restored can be variously modified in consideration of the user's temperature and a temperature change according to the point of time when a user puts clothes such as socks on, or other various environmental conditions.

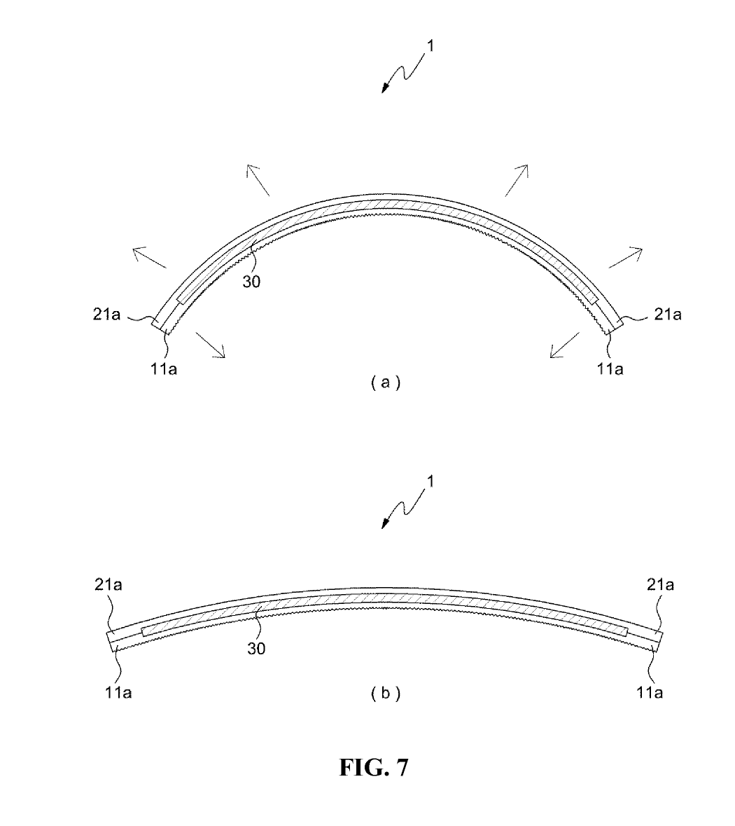

When the core 30 expands, as described above, the entire nail deformity correction device 1 including the core 30 is expanded and restored with the core 30, as shown in (b) of FIG. 7. That is, the nail deformity correction device 1 is attached to a fingernail/toenail in the bending state, as in (a) of FIG. 7, and when the core 30 is restored, the device expands and corrects the fingernail/toenail, as shown in (b) of FIG. 7. The contact portions (see 11 and 21 of FIG. 1), particularly the curvature-maintaining portions 11a and 21a expanding outward at the ends of the core 30 resist the transformation of the core 30, as described above, so the nail deformity correction device 1 maintains a predetermined curvature, as shown in (b) of FIG. 7.

That is, as described above, even if the core 30 memorizes a shape to expand completely straight at a set temperature, the entire nail deformity correction device 1 maintains a predetermined curvature by the resistance of the curvature-maintaining portions 11a and 21a positioned outside the core 30 in the expansion direction of the core 30, as shown in (b) of FIG. 7. Accordingly, it is possible to provide a correction force at a level suitable for appropriately expanding and correcting an affected part of a user without a pain due to excessive pressure on the affected part of the user.

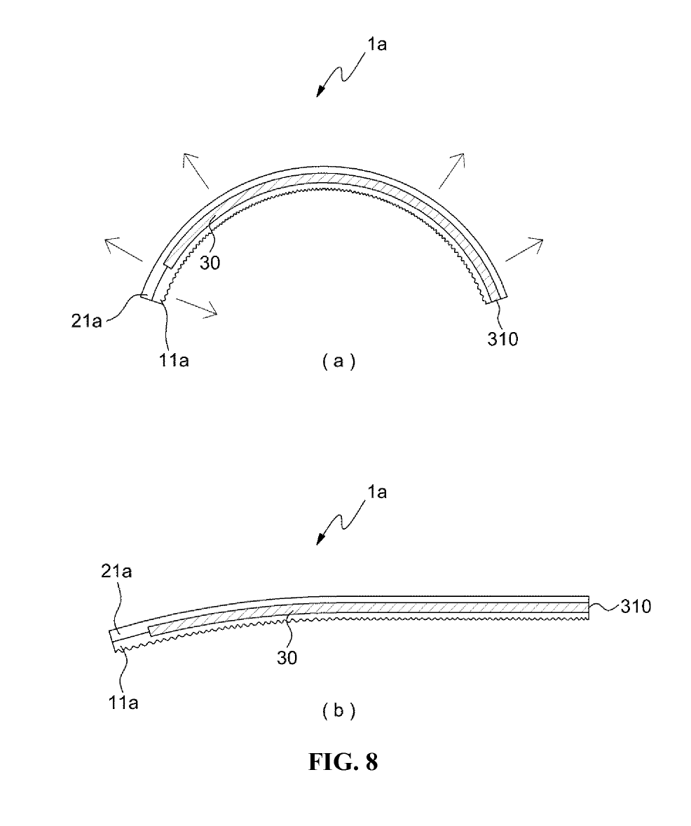

As shown in FIG. 8, when the exposing portion 310 is formed at a portion of the core 30, as in the modified example described above, the portion that generates resistance such as the curvature-maintaining portions 11a and 21a is minimized around the exposing portion 310, so it easily expands, but resistance is relatively large at the portion where the exposing portion 310 is not formed, so the curvature can be maintained. That is, as in (a) of FIG. 8, when the nail deformity correction device 1a having the exposing portion 310 is bent and attached to a fingernail/toenail and the core 30 expands due to a temperature change, as shown in (b) of FIG. 8, the exposing portion 310 and the curvature-maintaining portions 11a and 21a may expand in different amounts, as shown in the figure. Accordingly, if necessary, it may be possible to apply different correction forces to an affected part of a fingernail/toenail with the nail deformity correction device 1a so that the affected part is selectively corrected.

Hereafter, a process of attaching a nail deformity correction device and correcting an affected part is described with reference to FIGS. 9 to 14.

As shown in FIG. 9, first, an adhesive 40 is applied to the attachment surface 130. The adhesive 40 may be easily applied to the attachment surface 130 of the first body by a nozzle C. The adhesive 40 is applied to the entire attachment surface 130 and it is preferable to uniformly apply the adhesive to not only the portion overlapping the core 30, but the portion overlapping the curvature-maintaining portions 11a and 21a outside the core 30.

The nail deformity correction device 1 with the adhesive 40 applied is attached to an affected part of a fingernail/toenail N, as shown in FIG. 10. The nail deformity correction device 1 can be easily attached to the fingernail/toenail N by the adhesive 40 and can be firmly attached to the fingernail/toenail N by the adhesive 40 being in close contact with the attachment surface (see 130 in FIG. 9). As described above, when prominences and depressions are formed on the attachment surface 130, the adhesive permeates in between the fine prominences and depressions, so attachment can be more easily achieved. Further, the contact portions (see 11 and 21 in FIG. 1) outside the core 30 and the curved portions (see 111 and 211 in FIG. 3) can increase the attachment force, as described above.

The nail deformity correction device 1 attached to the fingernail/toenail N is bent in a shape corresponding to the deformed affected part of the fingernail/toenail N. The nail deformity correction device 1 is made of a material having elasticity and plasticity, so it can easily bend to correspond to the shape of the fingernail/toenail N. Further, since it is firmly attached by the adhesive 40, it is easily fixed to the surface of the fingernail/toenail N in the bending shape. The initial state when the nail deformity correction device 1 is attached may be a state in which the temperature where the core 30 is restored, that is, a set temperature is not reached yet.

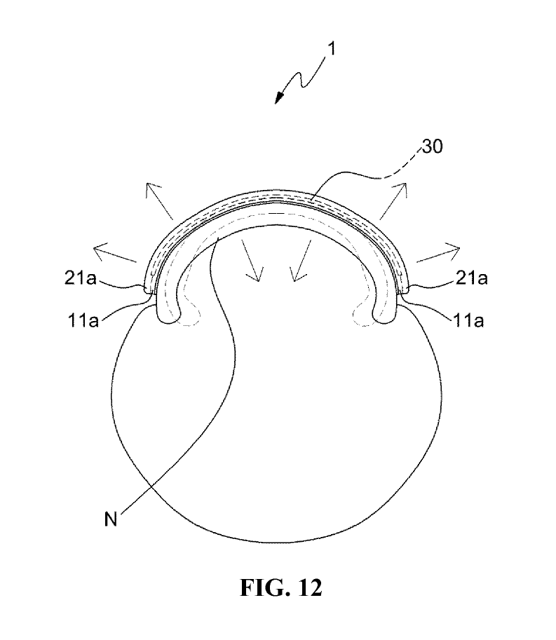

When the temperature around the fingernail/toenail N reaches the set temperature, the core is restored 30 and the nail deformity correction device 1 is expanded, as shown in FIG. 12. Accordingly, a correction force is applied to the fingernail/toenail N and the affected part is expanded along the nail deformity correction device 1 and corrected. As described above, the curvature of the nail deformity correction device 1 is maintained by the contact portions, particularly, the curvature-maintaining portions 11a and 21a, so it is possible to apply a correction force in the optimal state in which a user does not feel a pain and the affected part is easily corrected. It is possible to apply a correction force at various levels considering the size and the degree of deformation of an affected part by adjusting the areas of the curvature-maintaining portions 11a and 21a and the restoration force of the core 30.

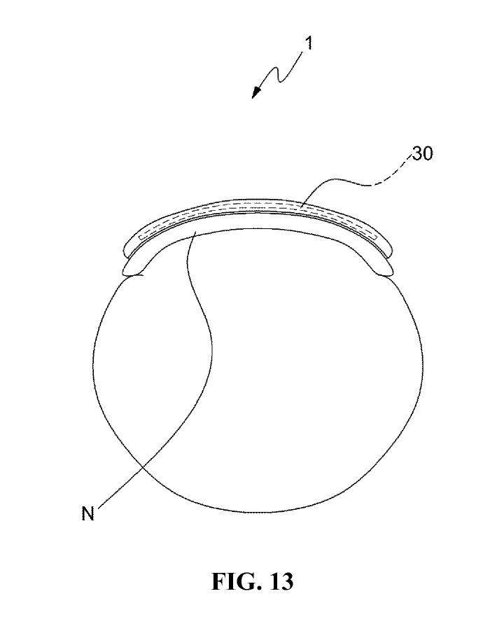

As described above, when the correction force is continuously applied, the fingernail/toenail N deformed, as shown in FIG. 13, is expanded and corrected in the normal state. The core 30 and the entire nail deformity correction device 1 can maintain a predetermined curvature, so it is possible to remove a side effect such as that pressure is abnormally applied even though correction is completed. A correction force at a very appropriate level is applied in this way, it is possible to correct the fingernail/toenail N very conveniently and effectively without a side effect such as a pain.

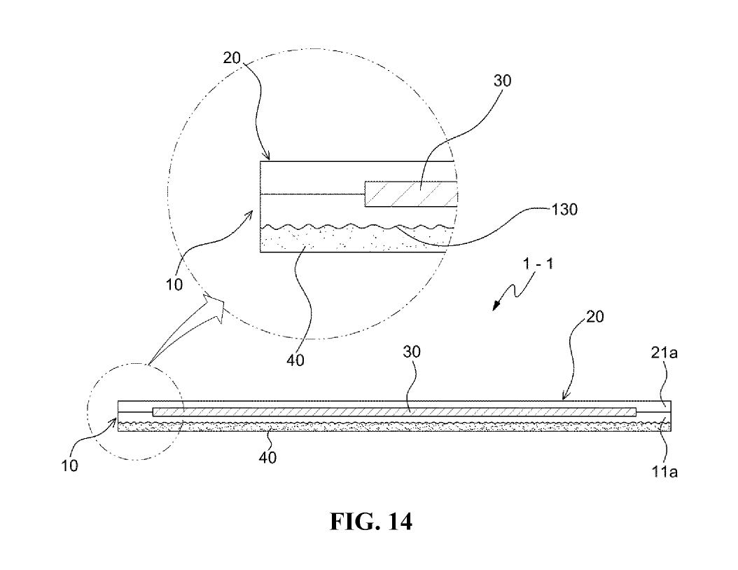

Hereinafter, a nail deformity correction device according to another exemplary embodiment of the present invention is described in more detail with reference to FIGS. 14 to 16. For simple and clear description, differences from the embodiment described above are described in priority and the configuration not specifically stated refers to that described above.

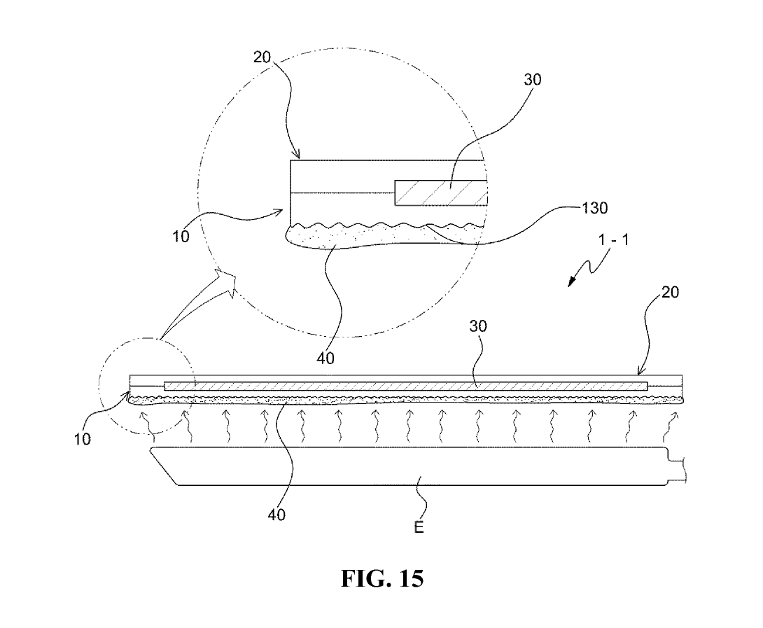

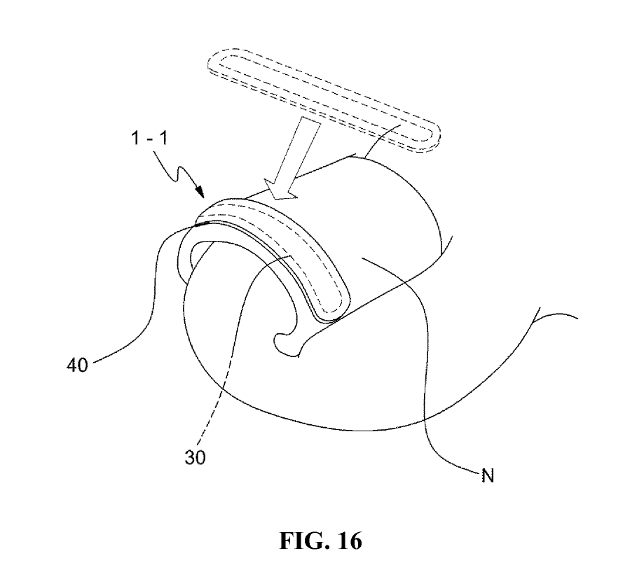

FIG. 14 is a cross-sectional view showing a nail deformity correction device according to another embodiment of the present invention, taken in the longitudinal direction and FIGS. 15 and 16 are views showing a process of mounting the nail deformity correction device of FIG. 14.

Referring to FIG. 14, in a nail deformity correction device according to another embodiment of the present invention, an adhesive 40 is hardened and fixed to an attachment surface 130 and includes thermoplastic resin that has fluidity when being heated. The adhesive 40 may be layered on the attachment surface 130.

The adhesive 40 including thermoplastic resin and layered on the attachment surface 130 is melted and has fluidity when it is brought close to a heat source, as shown in FIG. 15. Accordingly, there is no need for preparing the adhesive 40 separately from the nail deformity correction device 1-1 and it is possible to make the nail deformity correction device 1-1 be attachable by just heating the attachment surface 130. The heat source, for example, may be a drier or a curling iron, or a heating device E that can easily use heat such as a lighter.

When the adhesive 40 has fluidity, the nail deformity correction device 1-1 is attached to the fingernail/toenail N. The temperature where the adhesive 40 has fluidity is set higher than the temperature where the core 30 is restored, that is, the set temperature described above, so the nail deformity correction device 1-1 can be restored in accordance with a temperature change, as described above, keeping firmly attached to the fingernail/toenail N. In this way, the deformed fingernail/toenail N can be easily corrected.

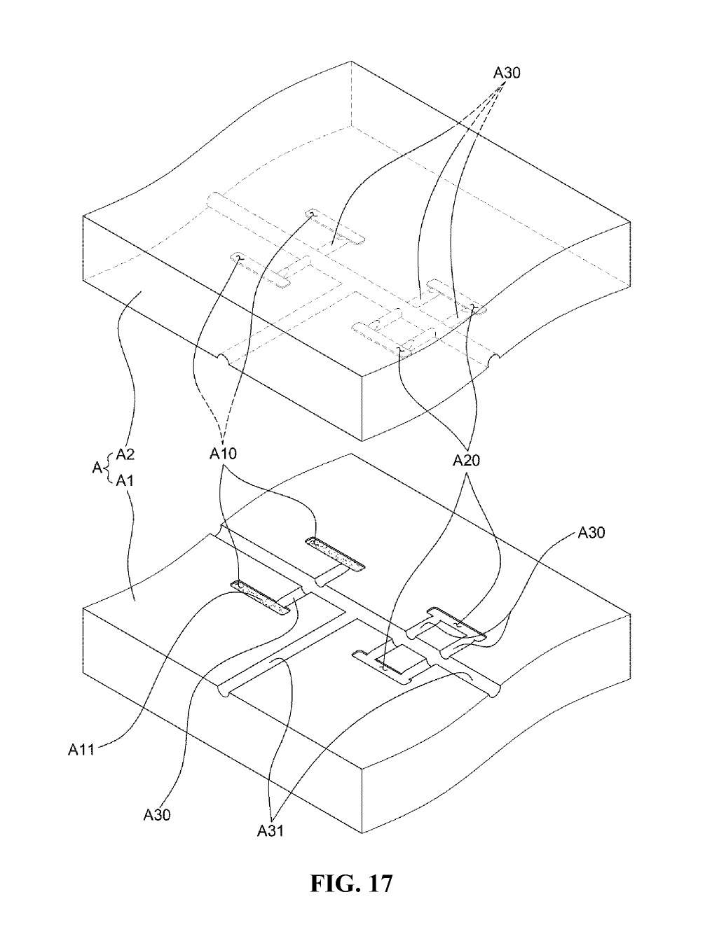

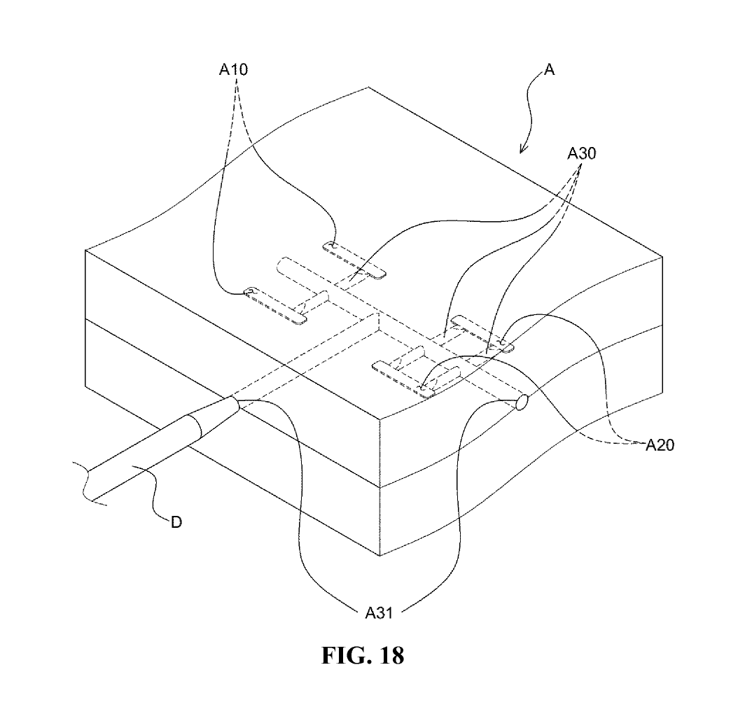

Hereinafter, a method of manufacturing a nail deformity correction device according to an embodiment of the present invention is described with reference to FIGS. 17 to 25.

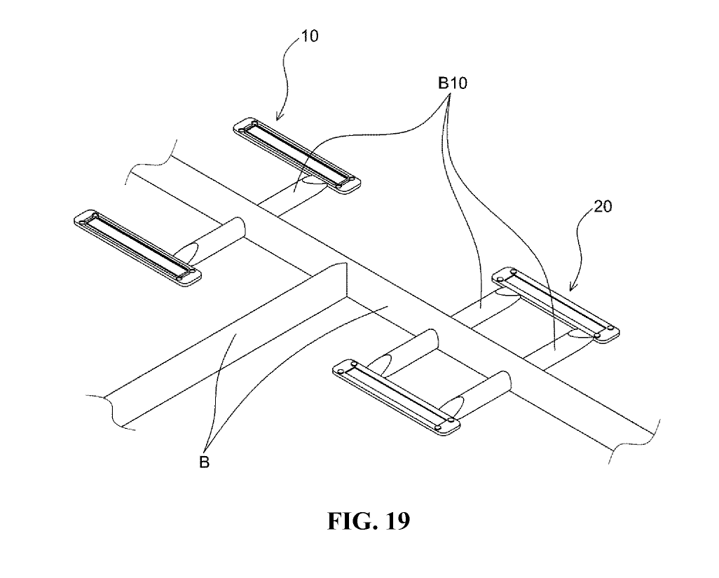

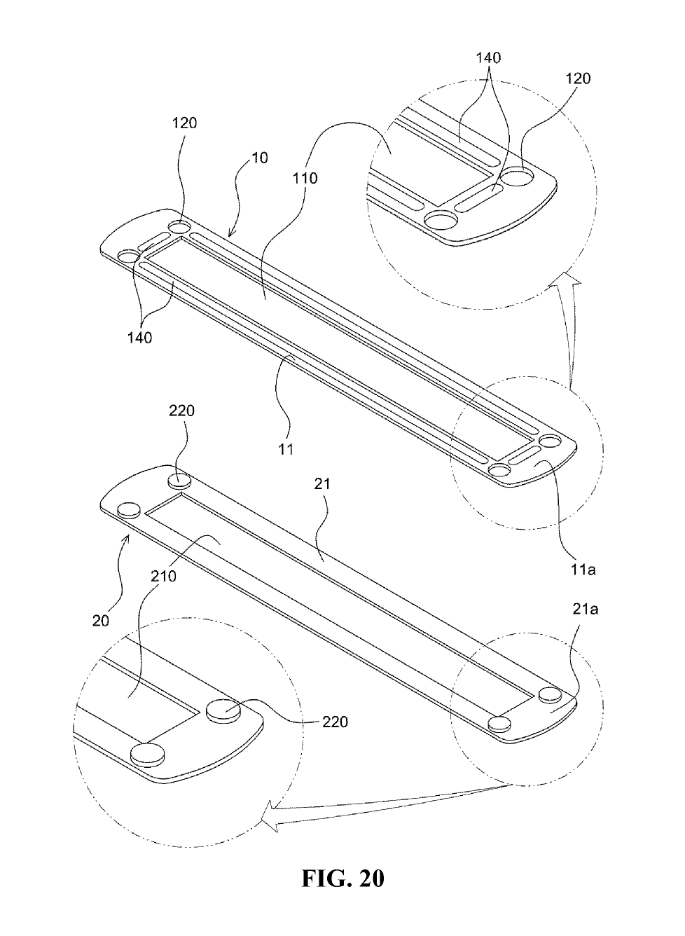

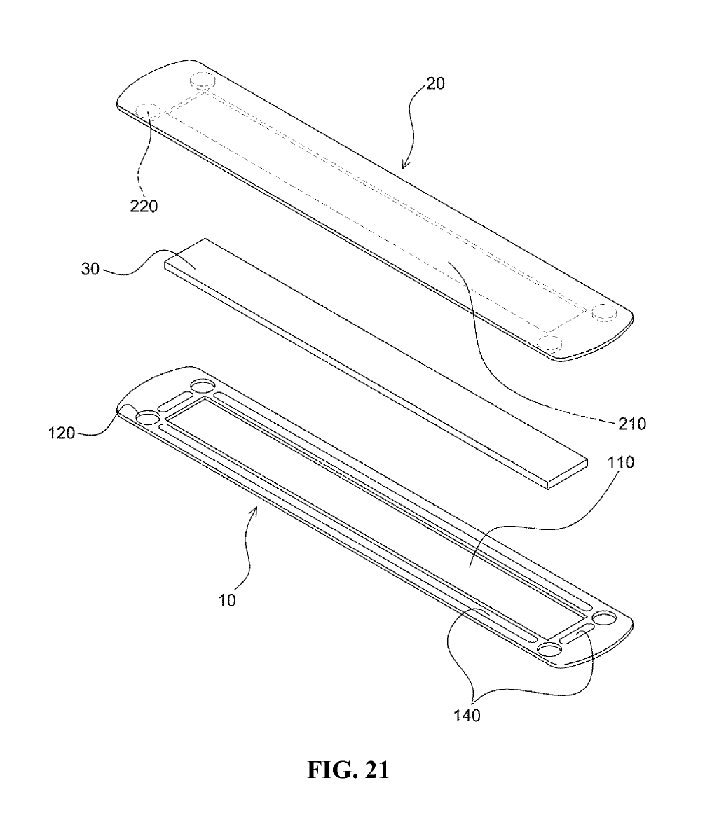

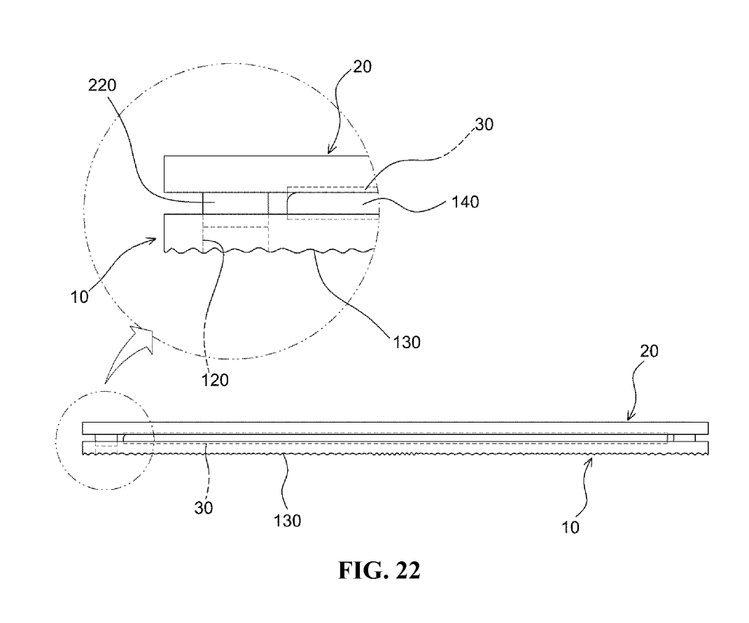

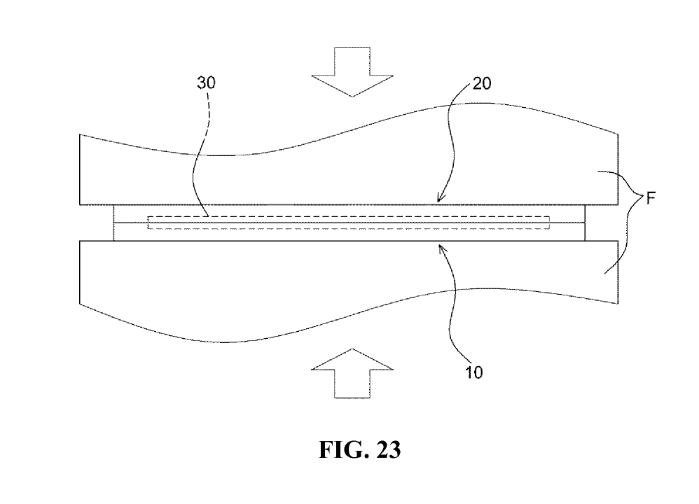

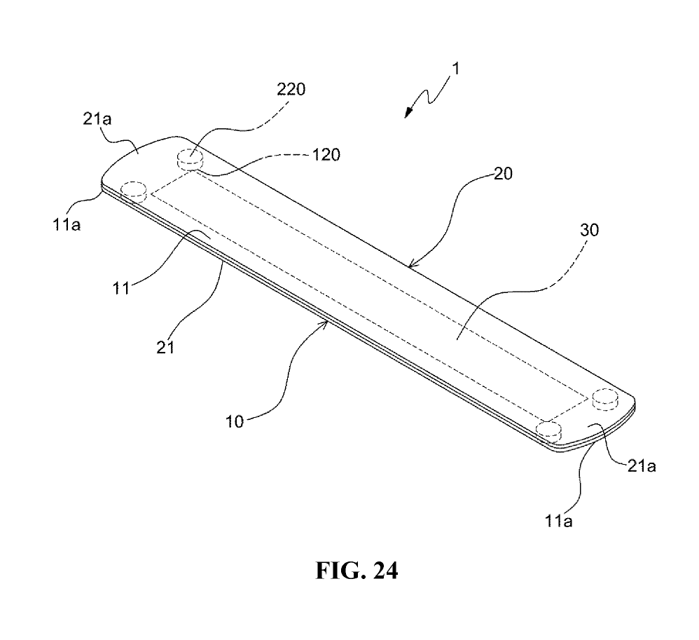

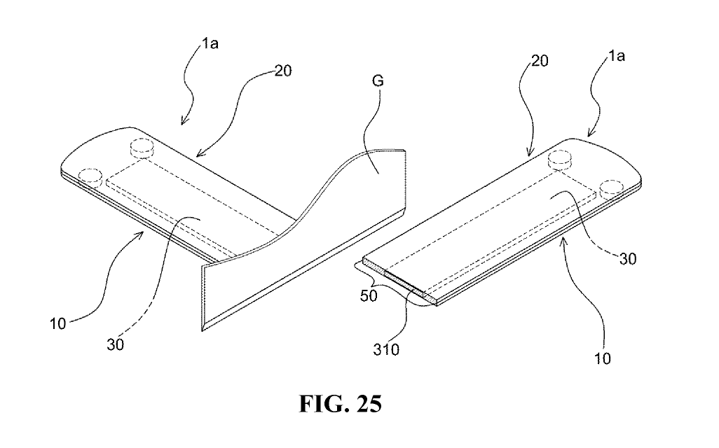

FIG. 17 is a perspective view showing an example of an available mold prepared in accordance with a method of manufacturing a nail deformity correction device according to an embodiment of the present invention, FIG. 18 is a perspective view showing a process of injection resin with the mold of FIG. 17 assembled, and FIG. 19 is a perspective view showing primary separation with a first body and a second body of the mold of FIG. 18 connected. Further, FIG. 20 is an enlarged perspective view the first body and the second body of FIG. 19 that are completely separated, FIGS. 21 and 22 are views showing a process of injecting and arranging a core in between the first body and the second body of FIG. 20, and FIG. 23 is a view showing a process of attaching the first body and the second body of FIG. 22. Further, FIG. 24 is a perspective view showing an example of a nail deformity correction device formed by attaching the first body and the second body of FIG. 23 with the core therebetween and FIG. 25 is a view showing a process of disassembling the nail deformity correction device of FIG. 24.

Referring to FIGS. 17 to 25, a method of manufacturing a nail deformity correction device according to an embodiment of the present invention includes: preparing a mold A having first grooves A10, second grooves A20, and at least one resin injection passage A30 connected to the first grooves A10 and the second grooves A20, as shown in FIG. 17 (step A); forming a first body (see 10 in FIG. 19) having a plate shape corresponding to the first grooves A10 and a second body 20 having a plate shape corresponding to the second grooves (see 20 in FIG. 19) by injecting and hardening resin in the first grooves A10 and the second grooves A20 through the resin injection passage A30, as shown in FIG. 18 (step B); separating the first body 10 and the second body 20 from the mold A and inserting and aligning a core 30 made of a shape memory material between the first body 10 and the second body 20, as shown in FIGS. 19 to 22 (step C); and attaching the first body 10 and the second body 20 with the core 30 between the first body 10 and the second body 20, as shown in FIGS. 23 and 24 (step D).

Further, after the attaching of the first body 10 and the second body with the core 30 between the first body 10 and the second body 20 (step D), the method may further include a step of forming a cutting surface 50 such that cross-sections of the first body 10, the second body 20, and the core 30 are all exposed, and then separating the first body 10, the second body 20, and the core 30 along the cutting surface 50 (step E). Through the steps, the core 30 is inserted between the first body 10 and the second body 20 and the nail deformity correction device (see 1 in FIG. 24) applying an appropriate correction force to an affected part can be easily manufactured. Hereafter, features of the steps (step A to step E) of the method of manufacturing a nail deformity correction device are described in detail with reference to the drawings.

First, the first step (step A) of the method of manufacturing a nail deformity correction device is described in detail with reference to FIG. 17. The step A of the method of manufacturing a nail deformity correction device may be a step of preparing the mold A. As shown in FIG. 17, the first groove A10 having a plate shape, the second grooves A20 having a plate shape corresponding to the first grooves A10, and at least one resin injection passage A30 connected to the first groove A10 and the second groove A20 are formed in the mold A. It is possible to more stably and easily form the first body (see 10 in FIG. 19) having elasticity and plasticity and a plate shape and the second body (see 20 in FIG. 19) having a plate shape by injecting resin into the mold A.

The mold A may be formed by detachably combining different metal mold frames A1 and A2, and the first grooves A10 and the second grooves A20 may be divided into pairs, respectively, on the mold sides facing each other of the different mold frames A1 and A2 constituting the mold A by being separably combined, as shown in FIG. 17. The mold sides may be the top of the lower mold frame A1 and the bottom of the upper mold frame A2 in FIG. 17.

When the different mold metal frames A1 and A2 are combined, with the mold sides in close contact with each other, as described above, the pairs of the first grooves A10 and the pairs of the second grooves A20 separated on the mold sides are combined, so complete first grooves (see A10 in FIG. 18) and second grooves (see A10 in FIG. 18) are formed. The first grooves A10 and the second grooves A20 are positioned in the mold A and can be supplied with resin only through the resin injection passages A30. The resin injection passages A30 are connected to channels A31 each having at least one end open to the outside of the metal frames A1 and A2, so resin can be easily supplied into the mold A from outside the mold A.

Further, as shown in FIG. 17, in the mold A, the resin injection passage A30 is positioned on the same plane as at least one of the first grooves A10 formed in the shape of a plate and the second grooves A20 formed in the shape of a plate, and a plurality of resin injection passages A30 may be connected in parallel with at least one of the first grooves A10 and the second grooves A20. Accordingly, when resin is supplied in parallel with at least one of the first grooves A10 and the second grooves A20 and hardened, at least of the first body 10 and the second body 20 can be stably maintained by runner connecting portions (see B10 in FIG. 19) connected in parallel with at least one of the first body (see 10 in FIG. 19) and the second body (see 20 in FIG. 19).

That is, the first body 10 and the second body 20 having flexibility to be elastically bent are formed by injecting resin into the plate-shaped first grooves A10 and second groove A20, and the runner connecting portions B10 connected in parallel with any one of the first body 10 and the second body 20 is formed by the resin injection passages A30 formed in parallel with the bodies, and transformation of at least one of the first body 10 and the second body 20 can be minimized and kept in parallel. Accordingly, it is possible to more stably form the first body 10 and the second body 20.

Further, the mold A may have contact surfaces A11 having prominences and depressions in the first grooves A10. Accordingly, when the resin is hardened in the first grooves A10, prominences and depressions can be formed on a surface of the first body 10 corresponding to the contact surfaces A11. An adhesive easily permeates in the prominences and depressions of the surface of the first body 10 where prominences and depressions are formed by the contact surfaces A11, so it can be more firmly attached to a fingernail/toenail. In this way, the first step of preparing the mold A is performed. Next, the second step (step B) of the method of manufacturing a nail deformity correction device is described in detail with reference to FIG. 18. The step B of the method of manufacturing a nail deformity correction device is a step of forming the first body 10 having a plate shape corresponding to the first grooves A10 and the second body 20 having a plate shape corresponding to the second grooves A20 by inserting and hardening resin in the first grooves A10 and the second groove A20 through the resin injection passages A30. As described above, when the mold A is formed by combining different mold frames, the first grooves A10, the second grooves A20, and the resin injection passages A30 connected with the grooves are arranged in the mold A and the resin injection passages A30 are connected to the outside of the mold A through the channels A31. Accordingly, as shown in FIG. 18, it is possible to easily inject liquid resin into the mold A through an injection pipe D and harden the resin.

The liquid resin may include polymeric resin that has elasticity and plasticity when being hardened. The liquid resin is supplied to the channels A31 from the injection pipe D and injected into the first grooves A10 and the second grooves A20 through the resin injection passages A30 connected with the channels A31. The resin is hardened in the first grooves A10 and the second grooves A20, thereby forming the first body (see 10 in FIG. 19) and the second body (see 20 in FIG. 19), and the resin is hardened in the resin injection passages A30, thereby forming the runner connecting portions (see B10 in FIG. 19) connected with the first body 10 and the second body 20. As described above, it is possible to easily form the first body 10 and the second body 20 by injecting and hardening liquid resin in the mold A. In this way, the second step of forming the first body 10 and the second body 20 in the mold A is performed.

Next, the third step (step C) of the method of manufacturing a nail deformity correction device is described in detail with reference to FIGS. 19 to 22. The step C of the method of manufacturing a nail deformity correction device is a step of separating the first body 10 and the second body 20 from the mold (see A in FIG. 18), and as shown in FIGS. 21 and 22, inserting and aligning the core 30 made of a shape memory material between the first body 10 and the second body 20. In particular, the first body 10 and the second body 20, which are primarily separated, are stably fixed by the runner connecting portion B10 formed by the resin hardened in the resin injection passages (see A30 in FIG. 18), as shown in FIG. 19. The runner connecting portions B10 are positioned in the same plane as at least one of the first body 10 having a plate shape and the second body 20 having a plate shape and connected in parallel with at least one of the first body 10 and the second body 20. Accordingly, at least one of the first body 10 and the second body 20 can be stably maintained in a flat state by the runner connecting portions B10. As described, since the flat state is maintained, more stable alignment is possible and later attachment can be easily performed by the stable alignment.

In FIG. 19, a pair of runner connecting portion B10 is formed in parallel with each of different second bodies 20. However, the runner connecting portions B10 are not limited thereto and may be connected in parallel with the first body 10. Further, the number of the runner connecting portions B10 is not limited to one pair, and if necessary, more runner connecting portions can be formed in parallel. The runner connecting portions B10 may be connected and fixed to runners B formed by the resin hardened in the channels (see A31 in FIG. 18), and the first body 10 and the second body 20 that are primarily separated can be stably supported by the runners B and the runner connecting portions B10.

The first body 10 and the second body 20 that are primarily separated from the mold are completely separated, as shown in FIG. 20, by removing the runners and the runner connecting portions. The first body 10 and the second body 20 that are separated from each other have the receiving portions 110 and 210 on the sides facing the core 30 (see 30 in FIG. 21), and the core 30 is inserted between the receiving portions 110 and 210, as shown in FIG. 21. Accordingly, the first body 10, the second body 20, and the core 30 are aligned in an easy attachment state.

Further, the first body 10 and the second body 20 include at least one of fusion grooves 120 formed outside the receiving portions 110 and 210 and fusion protrusions 220 formed outside the receiving portions 110 and 210, as shown in FIGS. 20 and 21, and the fusion grooves 120 and the fusion protrusions 220 can be combined and aligned with each other, as shown in FIG. 22.

Further, at least one of the first body 10 and the second body 20 may include fusion portions 140 elongated along the edge of the receiving portions 110 and 210. The fusion portions 140, for example, may protrude from the surface of at least one of the first body 10 and the second body 20. Accordingly, as shown in FIG. 22, when the first body 10, the second body 20, and the core 30 are aligned, the fusion portion 140 is positioned between the first body 10 and the second body 20.

In this specification, aligning means that the first body 10, the second body 20, and the core 30 are aligned to be easily attached in a state right before attachment is started, as shown in FIGS. 21 and 22. Aligning may be to change the alignment state such that the receiving portion 110 of the first body 10 and the receiving portion 210 of the second body 20 face each other, insert the core 30 between the different receiving portions 110 and 210, and insert the fusion protrusions 220 partially in the fusion grooves 120, as shown in FIG. 22, by primarily physically press them.

In this state, the linear fusion portions 140 are positioned between the first body 10 and the second body 20, as described above. In this way, the third step of separating the first body 10 and the second body 20 and inserting and aligning the core 30 between the bodies is performed.

Next, the fourth step (step D) of the method of manufacturing a nail deformity correction device is described in detail with reference to FIGS. 23 to 24. The fourth step in the method of manufacturing a nail deformity correction device is a step of attaching the first body 10 and the second body 20 with the core 30 between the first body 10 and the second body 20. The first body 10, the second body 20, and the core 30 are aligned, as described above, and then inserted in a fusion device F, as shown in FIG. 23, and the fusion device F can attach the first body 10 and the second body 20 to each other by applying heat and pressure. The attachment may be achieved by melting the fusion grooves 120 and the fusion protrusions 220 fitted in the fusion grooves 120, as shown in FIG. 24, and then bonding the first body 10 and the second body 20 to each other, and may be achieved by melting the linear fusion portions (see 140 in FIGS. 21 and 22) and bonding the first body 10 and the second body 20 to each other. That is, the first body 10 and the second body 20 may be attached to each other by thermal fusion that melts the contact portion with the core 30 therebetween or at least a portion of the contact surface with heat and then bonding it.

The thermal fusion is to directly melt an object with heat and then bond it, in which the heat applied to the object is transmitted by conduction through a heated tool, or applied directly to a corresponding portion by a laser, or internally generated by vibration energy such as a ultrasonic wave, that is, it can be supplied in various ways. Accordingly, the first body 10 and the second body 20 are inserted in the fusion device F in close contact with each other, but it is just an example, and it is possible to apply heat effectively in a contact or non-contact type to the first body 10 or the second body 20 using various devices having various shapes. Through this fusion, the nail deformity correction device 1 with the core 30 between the first body 10 and the second body 20 is manufactured, as shown in FIG. 24.

According to the nail deformity correction device 1 manufactured as described above, the core 30 is made of a shape memory material and can be restored to expand at a set temperature and the first body 10 and the second body 20 made of resin having elasticity and plasticity are expanded together, so a deformed fingernail/toenail can be corrected. The first body 10 can be firmly bonded by an adhesive between the attachment surface (see 130 in FIG. 22) and a fingernail/toenail.

In particular, the first body 10 and the second body 20 can at least partially have the contact portions 11 and 21 directly brought in close contact with each other by an attachment method such as fusion described above, outside the core 30, to resist restoration of the core 30. Accordingly, the nail deformity correction device 1 can maintain a predetermined curvature and apply a correction force at an appropriate level, which is not excessive, even in the expanded state. The contact portions 11 and 21 have the curvature-maintaining portions 11a and 21a extending outward at the ends of the core 30, so the curvature of the nail deformity correction device 1 can be more effectively maintained at an appropriate level.

Referring to FIG. 25, the nail deformity correction device 1a may be manufactured in a modified state by the addition step E (step E). As shown in the figure, after the first body 10 and the second body 20 are attached with the core 30 therebetween, the cutting surface 50 can be formed such that the cross-sections of the first body 10, the second body 20, and the core 30 are all exposed, using a cutter G. Accordingly, the first body 10, the second body 20, and the core 30 are separated along the cutting surface 50, so it can be changed in a shape having the cutting surface 50, as shown in the figure. The nail deformity correction device 1a modified as described above includes the exposing portion 310 that exposes a portion of the core 30 to the outside, so the other side having the exposing portion 310 and the opposite side can be restored in different degrees, and accordingly, correction forces having different magnitudes can be selectively applied to a fingernail/toenail. In this way, the nail deformity correction device 1a with the core 30 inserted between the first body 10 and the second body 20 can be easily manufactured.

According to the present invention, it is possible to effectively correct a fingernail/toenail by applying a correction force at an appropriate level to an affected part of the fingernail/toenail. Accordingly, it is possible to satisfactorily solve problems such as that a pain of a user is caused by unnecessarily excessive pressure from a correction device or a correction effect cannot be achieved by an insufficient correction force in the related art, and to efficiently perform correction work. Further, an affected part receives an appropriate correction force at an appropriate temperature level and is very easily corrected and it is possible to achieve a useful effect that correction is performed by a correction force at an appropriate level corresponding to changes in position or size of an affected part. Furthermore, according to a manufacturing method of the present invention, it is possible to very easily manufacture the nail deformity correction device applying a correction force at an appropriate level.

While the present invention has been described with respect to the specific embodiments, it will be apparent to those skilled in the art that various changes and modifications may be made without departing from the spirit and scope of the invention as defined in the following claims.

What is claimed is:

1. A nail deformity correction device, comprising:
a first body having a plate shape, elastically bent by an external force, and being attachable to the surface of a fingernail/toenail;
a second body having a plate shape, overlapping the first body, fixed to the first body, and elastically bending with the first body;
a core inserted between the first body and the second body and restored into a memorized shape in accordance with a temperature change; and
contact portions formed by bringing portions of the first body and the second body in close contact with each other outside the core,
wherein the first body, the second body, and the core expand and correct a deformed portion of the fingernail/toenail, and at least a portion of the contact portion resists restoration of the core.

2. The nail deformity correction device of claim 1, wherein the contact portions have curvature-maintaining portions expanding outward from ends of the core.

3. The nail deformity correction device of claim 2, wherein the core memorizes a shape to expand straight in a longitudinal direction at a set temperature and the curvature-maintaining portions are adjusted to maintain a predetermined curvature against transformation of the core.

4. The nail deformity correction device of claim 3, wherein the curvature of the core increases in proportion to the area of the curvature-maintaining portions.

5. The nail deformity correction device of claim 1, wherein the contact portions have curved portions convexly protruding outward at ends, and the ratio ‘b/a’ between a straight line ‘a’ connecting the start point and the end point of the curved portions and a perpendicular line ‘b’ from the top of the curved portions to the straight line ‘a’ is less than ½.

6. The nail deformity correction device of claim 1, wherein the contact portions have a shape in which convex portions and concave portions are continuously connected.

7. The nail deformity correction device of claim 1, wherein the first body is bonded to the fingernail/toenail by an adhesive and an attachment surface being in close contact with the adhesive is formed on the side of the first body facing the fingernail/toenail.

8. The nail deformity correction device of claim 7, wherein the attachment surface has prominences and depressions thereon.

9. The nail deformity correction device of claim 7, wherein the adhesive is hardened and fixed to the attachment surface and includes thermoplastic resin that has fluidity when being heated.

10. The nail deformity correction device of claim 1, wherein the core is made of a shape memory alloy that memorizes the shape to expand straight at 40° C.

11. The nail deformity correction device of claim 1, wherein the first body and the second body include polymeric resin having elasticity and plasticity.

12. The nail deformity correction device of claim 1, wherein the first body and the second body each have a receiving portion formed on the surface facing the core and the core is inserted between the receiving portions, at least one of fusion grooves and fusion protrusions is formed outside the receiving portions and the fusion grooves and the fusion protrusions formed outside the different receiving portions are combined with each other, and the first body and the second body are melted and fixed.

13. The nail deformity correction device of claim 1, wherein a portion of the core has an exposing portion that is exposed to the outside.

14. The nail deformity correction device of claim 13, wherein the contact portions extend from an end of the core and the exposing portion is formed at the other end of the core.

15. A method of manufacturing a nail deformity correction device, comprising:
(A) preparing a mold having first grooves formed in a plate shape, second grooves formed in a plate shape corresponding to the first grooves, and at least one resin injection passage connected to the first grooves and the second grooves;
(B) forming a first body having a plate shape corresponding to the first grooves and a second body having a plate shape corresponding to the second grooves by injecting and hardening resin in the first grooves and the second grooves through the resin injection passage;
(C) separating the first body and the second body from the mold and inserting and aligning a core made of a shape memory material between the first body and the second body; and
(D) attaching the first body and the second body with the core between the first body and the second body.

16. The method of claim 15, wherein the resin injection passages in the step (A) and the step (B) are positioned in the same plane as at least one of the first grooves formed in a plate shape and the second grooves formed in a plate shape and connected in parallel with at least one of the first grooves and the second grooves.

17. The method of claim 15, wherein in the step (A), the first grooves and the second grooves are formed in separate pairs on mold surfaces facing each other of different mold frames constituting the mold by being separably combined.

18. The method of claim 15, wherein in the step (C), the first body and the second body each have a receiving portion formed on the sides facing the core and the core is inserted between the different receiving portions.

19. The method of claim 15, wherein in the step (C), at least one of the first body and the second body further has linear fusion portions extending along the edge of the receiving portion and the fusion portions are positioned between the first body and the second body.

20. The method of claim 15, further comprising a step of forming a cutting surface such that cross-sections of the first body, the second body, and the core are all exposed, and separating the first body, the second body, and the core along the cutting surface, after the step (D).
